(19) United States
(12) Patent Application Publication
(10) Pub. No.: US 2009/0124897 A1
(43) Pub. Date: May 14, 2009

(54) Light-Directed Method for Detecting and Aiding Further Evaluation of Abnormal Mucosal Tissue

(75) Inventor: Douglas D. Burkett, Chandler, AZ (US)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: ZILA BIOTECHNOLOGY, INC., Phoenix, AZ (US)

(21) Appl. No.: 11/576,335

(22) PCT Filed: Sep. 30, 2004
(86) PCT No.: PCT/US04/32420

§ 371 Date: Apr. 25, 2007

(51) Int. Cl. A61B 6/00; A61B 5/00

(52) U.S. Cl. 600/431; 600/476

(57) ABSTRACT

In a light-directed method of identifying abnormal mucosal tissue, any suspect sites revealed by a light that selectively aids in visualizing abnormal tissue are marked with a dye facilitate further evaluation of the suspect tissue.

This invention relates to methods for detecting and aiding the evaluation of abnormal mucosal tissue.

In a more specific respect, the invention concerns a light-directed method, in which mucosal tissue indicated as abnormal by illuminating a gross anatomical area with light that selectively enhances visualization of abnormalities and before discontinuing illumination by selective light, any suspect abnormal sites so-located are then marked with a tissue marking agent, to assist in further evaluation of these abnormalities by any of a variety of techniques, to determine whether such suspect sites have serious pathology.

In still another and more particular respect the invention relates to such a method, in which the marking agent contains a staining dye which selectively indicates whether such a suspect site has serious pathology.

### BACKGROUND OF THE INVENTION

Patients who delay in obtaining a cancer consultation for at least two months have significantly higher relative hazards of death than do patients with a shorter delay. Thus, if patients are more regularly subjected to effective cancer screening, the mortality risks of cancer would be reduced.

Abnormal mucosal tissue, which may harbor tumor phenotypes, and which may indicate the presence of or the eventual development of invasive cancer can be visually identified and located in realtime in vivo using selective light examinations, which are admirably suited for rapid and inexpensive screening carried out as an adjunct to routing dental examinations. Illustratively, for example, U.S. Pat. Nos. 5,179,938 and 5,329,938, incorporated herein by reference, describe instruments equipped with a chemiluminescent light source which radiates in the visible green, blue and red spectrums, with spectral peaks at 450, 550 and 580 nm. Under such illumination, with normal ambient light suppressed, abnormal mucosal tissue appears white. Illustratively, such selective light devices for practicing such in vivo examinations are commercially available under the registered trademark VIZILITE® from Zila Pharmaceuticals, Inc., Phoenix, Ariz., USA.

Once an abnormal mucosal site has been located by selective light-directed screening, further diagnostic/prognostic procedures are then indicated to determine whether the suspect abnormal site has serious pathology. For example, biopsy of the suspect site, followed by conventional histological procedures or more recently developed “molecular analysis” techniques, are then performed to determine whether the abnormal tissue is cancerous or is in the progression pathway to likely develop invasive cancer. These molecular analysis techniques are disclosed in my International Application PCT/US02/32073 (WP 03/057918 A1), incorporated herein by reference.

While the selective light examinations can successfully identify and locate suspect mucosal tissue sites, the fact that the examination reveals these sites in real time, i.e., only while the selective light is directed onto the mucosal tissues, may make it difficult, after the selective light illumination is discontinued, to again locate and/or delineate such abnormal tissue sites for the purpose of further visual evaluation and, if indicated, conducting such further diagnostic/prognostic procedures, e.g., excision of histology or molecular analysis tissue samples of the abnormal tissue and/or of neighboring normal tissue, for comparison with the abnormal tissue.

To minimize such difficulty in locating the abnormal and/or normal tissue, it would be highly desirable to provide a selective light-directed diagnostic/prognostic method which incorporates a procedure for marking the location or delineating the abnormal tissue while the selective light is still directed upon the tissue, such that the abnormal tissue remains apparent after the selective light illumination is discontinued.

### BRIEF DESCRIPTION OF THE INVENTION

Briefly, I provide a light-directed method for detecting abnormal mucosal tissue which includes the steps of locating suspect mucosal tissue sites in a gross anatomical area, by illuminating the tissue with light that selectively enhances visualization of mucosal abnormalities, and marking such suspect sites to assist in further evaluation thereof, by applying a marking agent comprising a tissue-staining dye to such suspect sites.

In a further embodiment, the invention includes the use of a marking agent dye that both marks and delineates the extent of such suspect tissue.

In still another embodiment of the invention, I use a marking agent dye which selectively marks abnormal tissue, which may be cancerous or precancerous.

In a more specific embodiment, the invention includes the step of applying the marking agent by using a swab carrying a liquid marking agent containing the marking agent dye.

In another specific embodiment, the invention includes the step of marking the abnormal tissue by rinsing the gross anatomical area with a liquid containing the marking agent dye, using a dye which itself selectively marks abnormal tissue.

In still another embodiment of the invention, I employ a marking agent dye to a suspect abnormal site which dye, itself, further indicates whether such site has serious pathology.

In a still further embodiment the invention relates to the use of a staining dye in formulating a marking agent for use by applying the dye to suspect tissue identified by illuminating the tissue in a gross anatomical area with light that selectively enhances visualization of mucosal abnormalities.

These and other embodiments of my invention will be apparent to those skilled in the art from the following detailed illustrative description.

### DETAILED DESCRIPTION OF THE INVENTION

The following examples illustrate the invention and enable those skilled in the art how to practice the invention and identify the presently preferred embodiments thereof. As such, this description is not a limitation on the scope of the invention, which is expressed only by the appended claims.

### Example 1

This example illustrates the step of detecting abnormal mucosal tissue with a light that aids in selectively visualizing abnormal tissue in the oral cavity.

A routine visual examination of the oral cavity is made, noting the presence of any lesions on the attached gingiva, the buccal mucosa, the floor of the mouth, the hard and soft palate and the dorsal, lateral and ventral tongue.

The patient is then instructed to rinse the mouth with a 1% acetic acid solution for up to one minute and then expectorate.

The chemiluminescent light source described in the Lonky U.S. Pat. No. 5,329,938, commercially available under the registered trademark VIZILITE®, is activated by bending the flexible outer capsule, breaking the brittle inner vial. The capsule is then shaken and it is inserted into the retractor.

The ambient lights are then dimmed.

The visual examination of the oral cavity is then repeated using the illumination provided by the light source, looking for lesions or other suspect tissue sites which appear white.

### Example 2

This example illustrates the step of marking any suspect tissue sites identified in Example 1, by applying a tissue-staining dye marking agent to the suspect sites.

A swab is saturated with a toluidine blue 0 dye substance. This dye substance is disclosed in my published International Application WO99/25388, and is commercially available under the trademark Zila Tolonium Chloride™.

While the selective light is still being applied to the oral cavity, the dye is applied with the swab directly to each of the suspect sites identified by the light examination. The dye marks the tissue at the suspect site, marking the tissue dark blue, thus preserving the location of such suspect sites after the selective light illumination of Example 1 is removed.

The marked locations can then be evaluated for the presence of serious pathology, e.g. cancer or precancer. For example, the marked sites can then be sampled by excision, e.g., by a standard punch biopsy, to obtain tissue for subsequent examination, e.g., by standard histology or molecular analysis.

### Example 3

Instead of the dye used in Example 2, other dyes which selectively stain suspect mucosal tissue in vivo are employed to mark the suspect sites identified in Example 1. For example, dyes such as those disclosed in my published International Applications Nos. WO02/03048 (methylene blue), WO2/202149 (rhodamine) and the dyes disclosed in the published International Applications by Pomerantz, WO97/26018 (certain other oxazine and thiazine dyes), and Tucci, WO93/08847 (toluidine blue O+peroxide) are employed. These published International Applications are incorporated herein by reference.

Similar results are obtained.

### Example 4

Instead of applying the marking agent with a swab, the patient is directed to rinse the oral cavity with a liquid solution of the dyes of Examples 2 and 3. Similar results are obtained.

### Example 5

After completing the procedures of Examples 1 and 2, 3 or 4, the patient is re-examined in two weeks, using the same procedures. This delay allows suspect tissue that may not have serious pathology, such as simple abrasions, cuts, non-serious lesions such as apthous ulcers, etc. to heal. If this re-examination reveals the same suspect sites, then the probability of serious pathology is increased.

### Example 6

The procedures of Examples 1-4 are repeated, except that the gross anatomical areas examined includes the anal and vaginal mucosal tissues.

Similar results are obtained.

### Example 7

The cationic supravital dyes employed in Examples 2 and 3 will selectively enter the mitochondria of cancerous and precancerous tissues. The marking of suspect mucosal tissue sites by these dyes is further indication of serious pathology, such that the delayed re-examination of Example 5 before tissue sampling for histology or molecular analysis may not be necessary.

I claim:

1. A light-directed method for detecting abnormal mucosal tissue, said method comprising the steps of:
(a) locating suspect abnormal mucosal tissue sites in a gross anatomical area, by illuminating said tissue in vivo with light that selectively enhances visualization of mucosal abnormalities; and
(b) marking such suspect sites to assist in further evaluation thereof, by applying a marking agent comprising a tissue-staining dye to such suspect abnormal tissue sites.

2. The method of claim 1 in which said marking agent both marks and delineates such suspect tissue sites.

3. The method of claim 1 in which said marking agent selectively marks abnormal tissue sites.

4. The method of claim 1, in which said marking agent is applied to the suspect abnormal sites by a swab carrying a liquid marking agent containing said dye.

5. The method of claim 1, in which said marking agent is applied to such suspect abnormal sites by rinsing said gross anatomical area with a liquid marking agent containing said dye.

6. The method of claim 3, in which the selective staining of a suspect abnormal sites indicates whether such site has serious pathology.

7. The use of a staining dye in formulating the marking agent of claim 1.
